SEQUENCE LISTING (18 sequences)

SEQ ID NO: 1 - protein, 358 aa, Spinacia oleracea L
  Fructose-1,6-bisphosphatase
AAVGEAATETKARTRSKYEIETLTGWLLKQEMAGVIDAELTIVLSSISLACKQIASLVQRAGISNLTGIQGAVNIQGEDQKKLDVVSNEVFSSCLRSSGRTGIIASEEEDVPVAVEESYSGNYIVVFDPLDGSSNIDAAVSTGSIFGIYSPNDECIVDSDHDDESQLSAEEQRCVVNVCQPGDNLLAAGYCMYSSSVIFVLTIGKGVYAFTLDPMYGEFVLTSEKIQIPKAGKIYSFNEGNYKMWDDKLKKYMDDLKEPGESQKPYSSRYIGSLVGDFHRTLLYGGIYGYPRDAKSKNGKLRLLYECAPMSFIVEQAGGKGSDGHQRILDIQPTEIHQRVPLYIGSVEEVEKLEKYLA

SEQ ID NO: 2 - DNA, 1074 bp, Spinacia oleracea L
  Fructose-1,6-bisphosphatase
gcagccgtaggagaggcggctacagaaacaaaggcaaggactagaagtaagtacgaaattgaaacactaacaggctggctgcttaaacaagaaatggcaggtgttattgatgctgaacttaccatcgttctttctagcatttcattggcttgtaaacaaattgcttccttggttcaacgagctggtatttctaacttgactggaattcaaggtgctgtcaatatccaaggagaggatcagaagaaacttgatgttgtctccaatgaggtgttttcgagctgcttgagatcgagtggaagaacaggaataatagcatcagaagaagaggatgtaccagtggcagtggaagagagttactctggaaactatattgttgtgtttgatccacttgatggttcatccaacattgatgcagctgtctccactggttccatctttggcatttatagccctaacgatgagtgcattgttgactctgatcacgacgatgagtcacagctaagtgcagaagaacagaggtgtgtagtgaatgtatgtcaaccaggggataacctattagcagcagggtattgtatgtactcaagctctgttatcttcgtacttacaattggtaaaggtgtgtatgcattcacattagatccaatgtatggtgaattcgtactcacttcagagaaaatccaaatcccaaaagctgggaagatctattcattcaatgaaggtaactacaaaatgtgggatgataaattgaagaagtacatggatgatcttaaagagccaggagagtcacagaaaccgtactcgtctcgttacatagggagtttagttggggactttcatagaacacttttatatggtgggatttatggttacccaagagatgcaaagagtaagaatgggaaattgaggcttttgtatgaatgtgcacctatgagttttattgttgaacaagctggtggtaaaggttctgatggtcatcaaagaattcttgacattcaacccaccgagatacatcaacgtgtgccactgtacatcgggagtgtggaggaagtagagaaattagagaagtacttagca

SEQ ID NO: 3 - protein, 333 aa, Spinacia oleracea L
  Sedoheptulose-1, 7-bisphosphatase
VNKAKNSSLVTKCELGDSLEEFLAKATTDKGLIRLMMCMGEALRTIGFKVRTASCGGTQCVNTFGDEQLAIDVLADKLLFEALNYSHFCKYACSEELPELQDMGGPVDGGFSVAFDPLDGSSIVDTNFSVGTIFGVWPGDKLTGVTGRDQVAAAMGIYGPRTTYVLALKDYPGTHEFLLLDEGKWQHVKETTEINEGKLFCPGNLRATSDNADYAKLIQYYIKEKYTLRYTGGMVPDVNQIIVKEKGIFTNVISPTAKAKLRLLFEVAPLGFLIEKAGGHSSEGTKSVLDIEVKNLDDRTQVAYGSLNEIIRFEKTLYGSSRLEEPVPVGAAA

SEQ ID NO: 4 - DNA, 999 bp, Spinacia oleracea L
  Sedoheptulose-1,7-bisphosphatase
gtgaacaaggcaaagaactcttcccttgtaaccaaatgtgaacttggtgacagtttggaggagttcctagcaaaggcaaccacagataaagggctgattagattgatgatgtgcatgggagaagcattaaggaccattggctttaaagtgaggactgcttcatgtggtggaactcaatgtgttaacacctttggagacgaacagcttgccattgatgtgcttgctgacaagcttcttttcgaggcattgaactattcacacttctgcaagtatgcttgttcagaagaactccctgagcttcaagatatgggaggccccgttgatggcggattcagtgtagcatttgacccccttgatggatccagcattgtcgataccaatttctcagttgggaccatattcggggtttggccaggtgacaagctaactggtgtaacaggcagagatcaagtggctgctgcaatgggaatttatggtcctaggactacttatgttctcgctcttaaggactaccctggcacccatgaatttcttcttcttgatgaaggaaagtggcaacatgtgaaagaaacaacagaaatcaatgaaggaaaattgttctgtcctggaaacttgagagccacttctgacaatgctgattatgctaagctgattcaatactatataaaagagaaatacacattgagatacactggaggaatggttcctgatgttaaccagatcatagtgaaggagaaaggtatattcacaaatgtaatatcacctacagccaaggcaaagttgaggttactgtttgaggtagctcctctagggttcttgattgagaaggctggtggtcacagcagtgagggaaccaagtctgtgttggacattgaagtcaaaaaccttgatgacagaacccaagttgcttacggctccttgaacgagatcatccgatttgagaagacactatacggatcctctaggctagaggagccagttcctgttggagctgctgct

SEQ ID NO: 5 - protein, 356 aa, Synechococcus
  fructose-1,6-bisphosphatase/sedoheptulose-1, 7-bisphosphatase fromSynechococcus PCC 7942
MEKTIGLEIIEVVEQAAIASARLMGKGEKNEADRVAVEAMRVRMNQVEMLGRIVIGEGERDEAPMLYIGEEVGIYRDADKRAGVPAGKLVEIDIAVDPCEGTNLCAYGQPGSMAVLAISEKGGLFAAPDFYMKKLAAPPAAKGKETSIKSATENLKILSECLDRAIDELVVVVMDRPRHKELIQEIRQAGARVRLISDGDVSAAISCGFAGTNTHALMGIGAAPEGVISAAAMRCLGGHFQGQLIYDPEVVKTGLIGESRESNIARLQEMGITDPDRVYDANELASGQEVLFAACGITPGLLMEGVRFFKGGARTQSLVISSQSRTARFVDTVHMFDDVKTVSLPLIPDPKWRPER

SEQ ID NO: 6 - DNA, 1312 bp, Synechococcus
  fructose-1,6-bisphosphatase/sedoheptulose-1, 7-bisphosphatase fromSynechococcus PCC 7942
atcgcaactaaagccagagatgtgaggaggggatccggcctttggtagactcaactgttggaatccccagaagcaatcatccgtaaggagtcaggacggcgtggagaagacgatcggtctcgagattattgaagttgtcgagcaggcagcgatcgcctcggcccgcctgatgggcaaaggcgaaaagaatgaagccgatcgcgtcgcagtagaagcgatgcgggtgcggatgaaccaagtggaaatgctgggccgcatcgtcatcggtgaaggcgagcgcgacgaagcaccgatgctctatatcggtgaagaagtgggcatctaccgcgatgcagacaagcgggctggcgtaccggctggcaagctggtggaaatcgacatcgccgttgacccctgcgaaggcaccaacctctgcgcctacggtcagcccggctcgatggcagttttggccatctccgagaaaggcggcctgtttgcagctcccgacttctacatgaagaaactggctgcacccccagctgccaaaggcaaagagacatcaataaagtccgcgaccgaaaacctgaaaattctctcggaatgtctcgatcgcgccatcgatgaattggtggtcgtggtcatggatcgtccccgccacaaagagctaatccaagagatccgccaagcgggtgcccgcgtccgtctgatcagcgatggtgacgtttcggccgcgatctcctgcggttttgctggcaccaacacccacgccctgatgggcatcggtgcagctcccgagggtgtgatttcggcagcagcaatgcgttgcctcggcgggcacttccaaggccagctgatctacgacccagaagtggtcaaaaccggcctgatcggtgaaagccgtgagagcaacatcgctcgcctgcaagaaatgggcatcaccgatcccgatcgtgtctacgacgcgaacgaactggcttcgggtcaagaagtgctgtttgcggcttgcggtatcaccccgggcttgctgatggaaggcgtgcgcttcttcaaaggcggcgctcgcacccagagcttggtgatctccagccagtcacggacggctcgcttcgttgacaccgttcacatgttcgacgatgtcaaaacggttagcctgccgttaattcctgatcccaaatggcggccggagcggtagaacgggtatagctcgatcgcttcggtcgttgtttttcagcgaatccatttgcgatcgcttttcaaacccttttttcgtcaaccttctttaaacggcctcatgcatctcgcagttgtcggctcagccatcggacagcaccgg

SEQ ID NO: 7 - DNA, 133 bp, Nicotiana tabacum
  psbA promoter
agcttctacatacaccttggttgacacgagtatataagtcatgttatactgttgaataacaagccttccattttctattttgatttgtagaaaactagtgtgcttgggagtccctgatgattaaataaaccaa

SEQ ID NO: 8 - DNA, 159 bp, Nicotiana tabacum
  rps16 terminator
agcttgaaattcaattaaggaaataaattaaggaaatacaaaaaggggggtagtcatttgtatataactttgtatgacttttctcttctatttttttgtatttcctccctttccttttctatttgtatttttttatcattgcttccattgaattactag

SEQ ID NO: 9 - DNA, 805 bp, Escherichia coli
  aadA
gatccatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataactgca

SEQ ID NO: 10 - DNA, 4591 bp, Artificial sequence
  synthetic construct
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagttggatttgctcccccgccgtcgttcaatgagaatggataagaggctcgtgggattgacgtgagggggcagggatggctatatttctgggagcgaactccgggcgaatttgaagcgcttggatacagttgtagggagggatccatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataactgcaggatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaatcaaattttgacttcttcttatctcttatctttgaatatctcttatctttgaaataataatatcattgaaataagaaagaagagctatattcgaagcttctacatacaccttggttgacacgagtatataagtcatgttatactgttgaataacaagccttccattttctattttgatttgtagaaaactagtgtgcttgggagtccctgatgattaaataaaccaagatctaaaaggagaaattaagcatgctctagatcgatgaattcgcccttccgaagcttgaaattcaattaaggaaataaattaaggaaatacaaaaaggggggtagtcatttgtatataactttgtatgacttttctcttctatttttttgtatttcctccctttccttttctatttgtatttttttatcattgcttccattgaattactagtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 11 - DNA, 51 bp, Artificial sequence
  synthetic construct
ccaagatctaaaaggagaaattaagcatgctctagatcgatgaattcgccc

SEQ ID NO: 12 - DNA, 142 bp, Nicotiana tabacum
  rrn promoter
ctagttggatttgctcccccgccgtcgttcaatgagaatggataagaggctcgtgggattgacgtgagggggcagggatggctatatttctgggagcgaactccgggcgaatttgaagcgcttggatacagttgtagggagg

SEQ ID NO: 13 - DNA, 390 bp, Nicotiana tabacum
  psbA terminator
gatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaatcaaattttgacttcttcttatctcttatctttgaatatctcttatctttgaaataataatatcattgaaataagaaagaagagctatat

SEQ ID NO: 14 - DNA, 5581 bp, Artificial sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatgagttgtagggagggatttatgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggggtaatgcgccaggtgccgtagctaatcgagtagctctagaagcatgtgtaaaagctcgtaatgaaggacgtgatcttgctcaggaaggtaatgaaattattcgcgaggcttgcaaatggagcccggaactagctgctgcttgtgaagtatggaaagagatcgtatttaattttgcagcagtggacgttttggataagtaaaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagaaaactaaatcaaaatctaagactcaaatctttctattgttgtcttggatcgcggccgcgctagcgtcgacgatccttaggattggtatattcttttctatcctgtagtttgtagtttccctgaatcaagccaagtatcacacctctttctacccatcctgtatattgtcccctttgttccgtgttgaaatagaaccttaatttattacttatttttttattaaattttagatttgttagtgattagatattagtattagacgagattttacgaaacaattatttttttatttctttataggagaggacaaatctcttttttcgatgcgaatttgacacgacataggagaagccgccctttattaaaaattatattattttaaataatataaagggggttccaacatattaatatatagtgaagtgttcccccagattcagaactttttttcaatactcacaatccttattagttaataatcctagtgattggatttctatgcttagtctgataggaaataagatattcaaataaataattttatagcgaatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcctattgaaaataccaatgaagatccaaatcgaaaagtgaaaaacattcatagttggaggaatcgtgacaattctagttgcagtaatgttgattatttattcggcgttaaagacattcggaatttcatctctgatgacacttttttagttagtgataggaatggagacagttattccatctattttgatattgaaaatcatatttttgagattgacaacgatcattcttttctgagtgaactagaaagttctttttatagttatcgaaactcgaattatcggaataatggatttaggggcgaagatccctactataattcttacatgtatgatactcaatatagttggaataatcacattaatagttgcattgatagttatcttcagtctcaaatctgtatagatacttccattataagtggtagtgagaattacggtgacagttacatttatagggccgtttgtggtggtgaaagtcgaaatagtagtgaaaacgagggttccagtagacgaactcgcacgaagggcagtgatttaactataagagaaagttctaatgatctcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 1434 bp, Nicotiana tabacum
  rbcL
atgtcaccacaaacagagactaaagcaagtgttggattcaaagctggtgttaaagagtacaaattgacttattatactcctgagtaccaaaccaaggatactgatatattggcagcattccgagtaactcctcaacctggagttccacctgaagaagcaggggccgcggtagctgccgaatcttctactggtacatggacaactgtatggaccgatggacttaccagccttgatcgttacaaagggcgatgctaccgcatcgagcgtgttgttggagaaaaagatcaatatattgcttatgtagcttaccctttagacctttttgaagaaggttctgttaccaacatgtttacttccattgtaggtaacgtatttgggttcaaagccctgcgcgctctacgtctggaagatctgcgaatccctcctgcttatgttaaaactttccaaggtccgcctcatgggatccaagttgaaagagataaattgaacaagtatggtcgtcccctgttgggatgtactattaaacctaaattggggttatctgctaaaaactacggtagagccgtttatgaatgtcttcgcggtggacttgattttactaaagatgatgagaacgtgaactcacaaccatttatgcgttggagagatcgtttcttattttgtgccgaagcactttataaagcacaggctgaaacaggtgaaatcaaagggcattacttgaatgctactgcaggtacatgcgaagaaatgatcaaaagagctgtatttgctagagaattgggcgttccgatcgtaatgcatgactacttaacggggggattcaccgcaaatactagcttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttgatttactgcgtgatgattttgttgaacaagatcgaagtcgcggtatttatttcactcaagattgggtctctttaccaggtgttctacccgtggcttcaggaggtattcacgtttggcatatgcctgctctgaccgagatctttggggatgattccgtactacagttcggtggaggaactttaggacatccttggggtaatgcgccaggtgccgtagctaatcgagtagctctagaagcatgtgtaaaagctcgtaatgaaggacgtgatcttgctcaggaaggtaatgaaattattcgcgaggcttgcaaatggagcccggaactagctgctgcttgtgaagtatggaaagagatcgtatttaattttgcagcagtggacgttttggataagtaa

SEQ ID NO: 16 - DNA, 705 bp, Nicotiana tabacum
  accD
aatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcctattgaaaataccaatgaagatccaaatcgaaaagtgaaaaacattcatagttggaggaatcgtgacaattctagttgcagtaatgttgattatttattcggcgttaaagacattcggaatttcatctctgatgacacttttttagttagtgataggaatggagacagttattccatctattttgatattgaaaatcatatttttgagattgacaacgatcattcttttctgagtgaactagaaagttctttttatagttatcgaaactcgaattatcggaataatggatttaggggcgaagatccctactataattcttacatgtatgatactcaatatagttggaataatcacattaatagttgcattgatagttatcttcagtctcaaatctgtatagatacttccattataagtggtagtgagaattacggtgacagttacatttatagggccgtttgtggtggtgaaagtcgaaatagtagtgaaaacgagggttccagtagacgaactcgcacgaagggcagtgatttaactataagagaaagttctaatgatctcga

SEQ ID NO: 17 - DNA, 21 bp, Artificial sequence
  synthetic construct
cgcggccgcgctagcgtcgac

SEQ ID NO: 18 - DNA, 7 bp, Artificial sequence
  Synthetic construct
aggaggu

(19) United States
(12) Patent Application Publication
(10) Pub. No.: US 2009/0044300 A1
(43) Pub. Date: Feb. 12, 2009

(54) Method For Improving Productivity of Plant By Chloroplast Technology

(76) Inventors: Akiho Yokota, Ikoma-shi (JP); Shigeru Shigeoka, Sakai-shi (JP); Ken-ichi Tomizawa, Kyoto (JP)

Correspondence Address: WENDEROTH, LIND & PONACK, L.L.P., 2033 K STREET N. W., SUITE 800, WASHINGTON, DC, 20006-1021, US

(21) Appl. No.: 10/591,752

(22) PCT Filed: Mar. 2, 2005
(86) PCT No.: PCT/JP2005/004037

§ 371 Date: Sep. 26, 2006

(30) Foreign Application Priority Data
Mar. 3, 2004 (JP) 2004-059513

(51) Int. Cl. A01H 5/00; C12N 15/00; C12N 1/00

(52) U.S. Cl. 800/317.3; 435/320.1; 435/317.1; 800/298

(57) ABSTRACT

An object of the present invention is to provide a transformed plant which has high photosynthesis activity, and has promoted growth and productivity as compared with a wild strain, and has no fear of diffusion of an introduced gene by pollens, by expressing a trait of a specified gene by chloroplast technology in a higher plant. According to the present invention, there is provided a transformed plant using a gene recombinant vector having an expression cassette for enhancing photosynthesis activity, containing a DNA fragment comprising a gene encoding a protein having fructose-1,6-bisphosphatase sedoheptulose-1,7-bisphosphatase activities between a nucleotide sequence complementary to the chloroplast gene rbcL and the chloroplast gene aacD.

### TECHNICAL FIELD

The present invention relates to a transformed plant which has high photosynthesis activity and is excellent, particularly in fixation of carbon dioxide.

### BACKGROUND ART

A plant performs photosynthesis, fixes carbon dioxide in the air, and synthesizes a sugar and an organic substance which become energy source for an organism. In a plant, a process of fixing carbon dioxide in the air and synthesizing a sugar from carbon dioxide is called the Calvin cycle. The Calvin cycle does not need light energy, and is classified into the following two stages. The first stage is a process in which 3-phosphoglyceric acid (PGA) is synthesized from ribulose-1,5-bisphosphate (RuBP) and carbon dioxide, and this is further reduced, thereby to synthesize glyceraldehyde-3-phosphate (GAP). The second stage is a process in which a part of synthesized GAP is used for synthesizing a sugar (photosynthesis product), and a remaining GAP is reproduced into RuBP via fructose-1,6-bisphosphate (FBP), fructose-6-phosphate (F6P), sedoheptulose-1,7-bisphosphate (SBP), sedoheptulose-7-phosphate (S7P), ribose-5-phosphate and the like. Thereupon, synthesis of PGA from RuBP, that is, uptake of carbon dioxide into the Calvin cycle is catalyzed by ribulose-1,5-bisphosphate carboxylase/oxygenase (hereinafter, abbreviated as Rubisco). In the second stage, aldolase [enzyme which reversibly catalyzes the reaction from GAP and dihydroxyacetone phosphate (DHAP) to FBP, and the reaction from DHAP and erythrose-4-phosphate (E4P) to SBP, respectively], fructose-1,6-bisphosphatase (FBPase; enzyme which catalyzes the reaction from FBP to F6P), sedoheptulose-1,7-bisphosphatase (SBPase; enzyme which catalyzes the reaction from SBP to S7P), and transketolase are responsible for a metabolic reaction as the rate-limiting enzyme.

Many enzymes which act in the Calvin cycle are present at a higher level than that required for maintaining a continuous reaction for fixing carbon dioxide, in some cases. However, it is known that although FBPase and SBPase are important rate-limiting enzymes in the Calvin cycle, its level is extremely lower as compared with other enzymes in the Calvin cycle (see, Miyagawa et al., Nature Biotechnology, 2001, vol. 19, p. 965-969).

For this reason, as a transgenic plant for enhancing photosynthesis ability, a vector having a promoter which permanently and specifically expresses a fructose-1,6-bisphosphatase gene (cy-FBPase gene) of a cytosol obtained from a mesophyll cell peculiar in plant leaves, and a transgenic plant transformed with the vector are reported (International Publication WO 98/18940).

In addition, a method for expressing FBP/SBPase derived from a cyanobacterium Synechococcus PCC7942 gene is reported. According to this method, it is known that a transformed plant has higher photosynthesis activity as compared with a wild strain, and its growth is promoted (see JP-A-253768/2000, Miyagawa et al., Nature Biotechnology, 2001, vol. 19, p. 965-969).

However, any of the aforementioned transformants is such that each gene is introduced into a leaf nuclear genome by introducing a plasmid constructed using a gene into Agrobacterium tumefaciens, and infecting a leaf disc with this. For this reason, a protein expressed from a gene introduced into a plant was transferred into a chloroplast with a low possibility.

In addition, introduction of a heterogeneous gene into a nuclear genome gives a fear that an introduced artificially modified gene is diffused into the environment by crossing or mating. Further, expression of the thus introduced gene is unstable, and an expression amount, consequently, the effect is greatly different every plant.

A higher plant chloroplast is present at the number of about 100 per one cell of an adult leaf, and 100 copies of a chloroplast genome are present per one chloroplast (see Archives of Biotechnology and Biophysics, 1996, vol. 334, p. 27-36; Bendich, A. J. BioEssays, 1987, vol. 6, p. 279-282).

This means that, if one copy of a foreign gene is inserted into a chloroplast genome, 10000 copies becomes to be present per cell in a transformant, and high expression of an introduced gene can be expected due to a large copy number (see Maliga, P. Trends in biotechnology, 1993, vol. 11, p. 101-107).

Further, since introduction of a gene into a chloroplast utilizes homologous recombination, positional effect seen upon insertion into a nucleus is not caused, and stable gene expression is performed. In addition, since a chloroplast is maternally inherited, it is thought that introduction of a gene into a chloroplast has many advantages, such as prevention of an introduced gene from flying into the environment via pollens.

An expression vector which can highly express a desired protein in a chloroplast, a transformed chloroplast transformed using the expression vect a plant having the transformed chloroplast are known. This expression vector is characterized in that it has a psbA promoter, and a ribosome-binding site upstream of a translation initiation point of a gene encoding a protein. This method is aimed at producing a protein having pharmacological activity, and a protein useful as a material for medicine industry, using a plant instead of production with microorganisms. In the Example, expression of the protein is confirmed in plants transformed using a gene of the green fluorescent protein (see Maliga, P. Trends in biotechnology, 1993, vol 11, p. 101-107)

However, in this reference, there is no description regarding improvement in photosynthesis activity or fixation of carbon dioxide in plants, and there is no description regarding FBPase or SBPase which is the rate-limiting enzyme of the Calvin cycle, or those genes.

### DISCLOSURE OF THE INVENTION

An object of the present invention is to produce a transformed plant which has higher photosynthesis activity as compared with the wild strain, and has promoted the growth, by expressing a gene of an enzyme involved in photosynthesis of higher plants, particularly, in the Calvin cycle. More particularly, the object is to introduce a gene of an enzyme which is rate-limiting in the Calvin cycle into chloroplast DNA, and produce a plant having transformed chloroplasts, photosynthesis ability of which is enhanced.

The present inventors have found that transformation technique which can assuredly express a protein having FBPase and/or SBPase in higher plant chloroplasts. In addition, a transformed plant not only has high photosynthesis activity, but also is grown into a plant having a greater plant body. The present invention has been completed by various further studies based on these findings.

That is, the present invention relates to:
(1) A gene recombination vector containing an expression cassette for enhancing photosynthesis activity, comprising a DNA fragment comprising a gene encoding a protein having FBPase and/or SBPase activities between a Rubisco large subunit gene and a acetyl CoA carboxylase subunit gene,
(2) The vector according to the above (1), wherein the protein having FBPase activity is any one of the followings;
(a) a protein comprising an amino acid sequence described in SEQ ID NO: 1 of Sequence Listing;
(b) a protein comprising an amino acid sequence in which one or several amino acids are deleted, substituted, added or inserted in SEQ ID NO: 1 of Sequence Listing, and having FBPase activity; and
(c) a protein having at least 60% or more homology to an amino acid sequence described in SEQ ID NO: 1 of Sequence Listing, and having FBPase activity,
(3) The vector according to the above (1), wherein the gene encoding a protein having FBPase activity is a gene comprising any one of the following DNAs;
(a) DNA comprising a nucleotide sequence described in SEQ ID NO: 2 of Sequence Listing;
(b) DNA comprising a nucleotide sequence in which one or several bases are deleted, substituted, added or inserted in SEQ ID NO: 2 of Sequence Listing, and encoding a protein having FBPase activity;
(c) DNA which hybridizes with DNA comprising a nucleotide sequence complementary to DNA comprising a nucleotide sequence described in SEQ ID NO: 2 of Sequence Listing under stringent conditions, and comprises a nucleotide sequence encoding a protein having FBPase activity; and
(d) DNA having at least 60% or more homology to DNA comprising a nucleotide sequence described in SEQ ID NO: 1 of Sequence Listing, and comprising a nucleotide sequence encoding a protein having FBPase activity,
(4) The vector according to the above (1), wherein the protein having SBPase activity is any one of the following proteins;
(a) a protein comprising an amino acid sequence described in SEQ ID NO: 3 of Sequence Listing;
(b) a protein comprising an amino acid sequence in which one or several amino acids are deleted, substituted, added or inserted in SEQ ID NO: 3 of Sequence Listing, and having SBPase activity; and
(c) a protein having at least 60% or more homology to an amino acid sequence described in SEQ ID NO: 3 of Sequence Listing, and having SBPase activity,
(5) The vector according to the above (1), wherein the gene encoding a protein having SBPase activity is a gene comprising any one of the following DNAs;
(a) DNA comprising a nucleotide sequence described in SEQ ID NO: 4 of Sequence Listing;
(b) DNA comprising a nucleotide sequence in which one or several bases are deleted, substituted, added or inserted in SEQ ID NO: 4 of Sequence Listing, and encoding a protein having SBPase activity;
(c) DNA which hybridizes with DNA comprising a nucleotide sequence complementary to DNA comprising a nucleotide sequence described in SDQ ID NO: 4 of Sequence Listing under stringent conditions, and comprises a nucleotide sequence encoding a protein having SBPase activity; and
(d) DNA having at least 60% or more homology to DNA comprising a nucleotide sequence described in SDQ ID NO: 4 of Sequence Listing, and comprising a nucleotide sequence encoding a protein having SBPase activity,
(6) The vector according to (1), wherein the protein having FBPase and SBPase activities is any one of the followings:
(a) a protein comprising an amino acid sequence described in SEQ ID NO: 5 of Sequence Listing;
(b) a protein comprising an amino acid sequence in which one or several amino acids are deleted, substituted, added or inserted in SEQ ID NO: 5 of Sequence Listing; and having FBPase and SBPase activities; and
(c) a protein having at least 60% or more homology to an amino acid sequence described in SEQ ID NO: 5of Sequence Listing; and having FBPase and SBPase activities,
(7) The vector according to the above (1), wherein the gene encoding a protein having FBPase and SBPase activities is a gene comprising any one of the following DNAs;
(a) DNA comprising a nucleotide sequence described in SEQ ID NO: 6 of Sequence Listing;
(b) DNA comprising a nucleotide sequence in which one or several bases are deleted, substituted, added or inserted in SEQ ID NO: 6 of Sequence Listing, and encoding a protein having FBPase and SBPase activities;
(c) DNA which hybridizes with DNA comprising nucleotide sequence complementary to a DNA comprising a nucleotide sequence described in SEQ ID NO: 6 of Sequence Listing under stringent conditions, and comprises a nucleotide sequence encoding a protein having FBPase and SBPase activities; and
(d) DNA having at least 60% or more homology to DNA comprising a nucleotide sequence described in SEQ ID NO: 6 of Sequence Listing, and comprising a nucleotide sequence encoding a protein having FBPase and SBPase activities,
(8) The vector according to any one of the above (1) to (7), wherein the expression cassette has a ribosome-binding site upstream of the translation initiation point of the DNA fragment comprising a gene encoding a protein having FBPase and/or SBPase activities,
(9) The vector according to the above (8), wherein the expression cassette has a promoter upstream of a ribosome-binding site, and a terminator downstream of DNA fragment comprising a gene encoding a protein having FBPase and/or SBPase activities,
(10) The vector according to the above (9), wherein the promoter and the terminator are a promoter and a terminator derived from tobacco chloroplasts, respectively,
(11) The vector according to any one of the above (1) to (10), wherein the Rubisco large subunit gene and the acetyl CoA carboxylase subunit gene are genes derived from tobacco, respectively,
(12) A recombinant gene vector comprising an expression cassette containing a DNA fragment comprising a gene encoding a protein having FBPase and/or SBPase activities between a tobacco-derived Rubisco large subunit gene and a acetyl CoA carboxylase subunit gene, having a ribosome-binding site upstream of the translation initiation point of the DNA fragment, having a tobacco-derived promoter between a Rubisco large subunit gene and a ribosome-binding site, and having a tobacco-derived terminator between the acetyl CoA carboxylase subunit gene and the DNA fragment,
(13) A transformed chloroplast characterized in that the vector described in any one of the above (1) to (12) is introduced into chloroplasts,
(14) A plant containing the transformed chloroplasts in the above (13),
(15) The plant according to the above (14), wherein the plant is tobacco, and
(16) A plant having 2-fold or higher FBPase activity compared to the original one, characterized in that a FBP/SBP gene is introduced into chloroplast genome of higher plants and expressed using a chloroplast transformation technique.

Also, the present invention relates to a process for producing a plant having transformed chloroplasts, comprising inserting a DNA fragment comprising a gene encoding a protein having FBPase and/or SBPase activities into a non-coding region between genes of chloroplast DNA.

The vector of the present invention can assuredly introduce a protein having FBPase and/or SBPase activities into higher plant chloroplasts. In a plant transformed with the vector of the present invention, since expression of a protein having FBPase and/or SBPase activities, which is a rate-limiting enzyme of the Calvin cycle, is enhanced, photosynthesis ability is enhanced as compared with the wild strain. As a result, in the transformed plant of the present invention, ability to synthesize sugars or starch can be enhanced as compared with the wild strain. In addition, the transformed plant of the present invention is tall, has a large area of leaves, has a thick stem, and the plant can grow rapidly. Therefore, cultivation of the transformed plant using the vector of the present invention can be a very effective means for producing a quickly growing, or a high yield plant.

In the transformed plant of the present invention, since a gene encoding a protein having FBPase and/or SBPase activities is introduced directly into the chloroplast genome rather than into the nuclear genome, there is no fear that the introduced gene is diffused through pollens. That is, there is no fear of environmental pollution that the pollen is scattered in a wide range via the wind or an insect, and this adversely influences on an animal and plant kingdom, for example, as in a plant in which a gene is introduced in a nucleus. In addition, expression is stable among transformants. In addition, the transformed plant of the present invention in which a gene is directly introduced into the chloroplast genome has enhanced ability to synthesize sugars or starches, and has a tall plant body and the large leaves compared with a plant transformed the gene into the nuclear genome, and can grow quickly with a high yield.

Since by utilizing recombinant DNA technology, photosynthesis which is the primary metabolic process in higher plants is improved, and thus their quick growth or high yields are made possible, the present invention can be an extremely important technique for responding to future food crisis.

In addition, in the transformed plant of the present invention, a rate-limiting enzyme of the Calvin cycle which plays an important role among photosynthesis, in particular, in fixation of carbon dioxide is enhanced. For this reason, since the transformed plant of the present invention has an enhanced rate of taking up carbon dioxide in the air, and can decrease the concentration of carbon dioxide in the air, cultivation of the plant can also contribute to suppression of global warming.

### BRIEF DESCRIPTION OF THE DRAWINGS

FIG. 1 is a view showing an expression vector pLD200-S.7942FBP/SBPase;

FIG. 2 is a view showing confirmation of gene introduction by PCR. In the figure, W denotes a wild strain;

FIG. 3 is a view showing confirmation of the introduced gene and the expressed protein in a plant 10 weeks after seeding. In the figure, W stands for the wild strain;

FIG. 4 is a view showing comparison of FBPase activity in an upper leaf and a lower leaf 10 weeks and 18 weeks after seeding. In the figure, the ordinate axis is for FBPase activity;

FIG. 5 is a view showing photosynthesis activity 10 weeks after seeding;

FIG. 6 is a view showing a growth rate. In the figure, the ordinate axis denotes the height (cm) of a plant;

FIG. 7 is a view showing plants 18 weeks after seeding; and

FIG. 8 is a view showing stems and roots in plants 18 weeks after seeding.

### BEST MODE FOR CARRYING OUT THE INVENTION

The protein having FBPase and/or SBPase activities used in the present invention is a protein which can be a rate-limiting enzyme of the Calvin cycle. The protein may have activity of any enzyme of FBPase or SBPase, or may have activities of both enzymes. In particular, in higher plants, a protein having enzyme activity of SBPase which can be pacemaker enzyme governing the rate of a series of reactions of the Calvin cycle as a whole, and a protein having both activities of FBPase and SBPase (hereinafter, abbreviated as FBP/SBPase) are preferable.

Examples, of the protein having FBPase activity include an amino acid sequence represented by SEQ ID NO: 1. In addition, examples of the protein having SBPase activity include an amino acid sequence represented by SEQ ID NO: 3. Examples of the protein exhibiting FBP/SBPase activities include an amino acid sequence of cyanobacterium-derived FBP/SBPase represented by SEQ ID NO: 5. The protein having FBPase and/or SBPase activities used in the present invention includes proteins having an amino acid sequence in which one or several amino acids are deleted, substituted, added or inserted in the aforementioned respective amino acid sequences, and each having FBPase activity, SBPase activity or FBP/SBPase activities. Further, the protein having FBPase and/or SBPase activities used in the present invention include a protein having at least 60% or more homology, preferably 80% or more homology, more preferably 90% or more homology, and furthermore preferably 95% or more homology to an amino acid sequence described in SEQ ID NO: 1, 3 or 5, each of which having FBPase activity, SBPase activity or FBP/SBPase activities.

As used herein, “homology” regarding an amino acid sequence is used to mean an extent of coincidence of amino acid residues constituting each sequence between sequences when the primary structures of proteins are compared.

In addition, as used herein, “one or several (around 2 to 6) amino acids are deleted, substituted, added or inserted” regarding an amino acid sequence means that a naturally-occurring number of amino acids are deleted, substituted, added or inserted by the well-known technological method such as a site-specific mutagenesis method.

The DNA fragment comprising a nucleotide sequence encoding a protein having FBPase and/or SBPase activities used in the present invention refers to DNA encoding each enzyme of FBPase, SBPase, and FBP/SBPase and DNA encoding a protein having an active site of the aforementioned each enzyme. Examples of the nucleotide sequence encoding a protein having FBPase activity include a DNA sequence represented by SEQ ID NO: 2. Examples of the nucleotide sequence encoding a protein having SBPase activity include a DNA sequence represented by SEQ ID NO: 4. Examples of the nucleotide sequence encoding a protein having FBP/SBPase activities include a DNA sequence represented by SEQ I NO: 6. The DNA fragment encoding a protein having FBPase and/or SBPase activities used in the present invention includes DNA which comprises a nucleotide sequence in which one or several bases are deleted, substituted, added or inserted in the aforementioned DNA sequence represented by SEQ ID NO: 2, 4 or 6, and encodes a protein having FBPase activity, SBPase activity, or FBP/SBPase activities. As used herein, “one or several bases are deleted, substituted, added or inserted” regarding a nucleotide sequence means that a naturally-occurring number (1 to several) of bases are deleted, substituted, added or inserted by the well-known technological method such as a site-directed mutagenesis method.

The DNA fragment encoding a protein having FBPase and/or SBPase activities used in the present invention includes DNA hybridizing with DNA comprising a nucleotide sequence complementary to each of DNA sequence shown in SEQ ID NO: 2, 4 or 6 under stringent condition, which also comprises a nucleotide sequence encoding a protein having FBPase activity, SBPase activity or FBP/SBPase activities. The DNA which can hybridize under stringent conditions means DNA which is obtained by using the aforementioned DNA as the probe, by such as the colony hybridization method, the plaque hybridization method or the Southern blot hybridization method. The stringent condition refers to the hybridizing condition of SSC solution of the salt concentration about 0.1 to 2-fold (a composition of SSC solution at 1-fold concentration comprises 150 mM sodium chloride, and 15 mM sodium citrate) at the temperature of about 65° C.

Further, the DNA fragment encoding a protein having FBPase and/or SBPase activities used in the present invention include DNA having at least 60% or more homology to each DNA sequence shown in SEQ ID NO: 2, 4 or 6, and also comprising a nucleotide sequence encoding a protein having FBPase activity, SBPase activity or FBP/SBPase activities. The DNA having homology refers to DNA having at least about 60% or more homology, preferably DNA having about 80% or more homology, more preferably DNA having about 90% or more homology, and furthermore preferably DNA having about 95% or more homology, under high stringent conditions. High stringent conditions refer to, for example, conditions where the sodium concentration is about 19 to 40 mM, preferably about 19 to 20 mM, and the temperature is about 50 to 70° C., preferably about 60 to 65° C. In particular, the conditions where the sodium concentration is about 19 mM and the temperature is about 65° C. is most preferable.

Hereinafter, a DNA fragment encoding a protein having FBPase and/or SBPase activities, as well as said hybridizing DNA and said DNA having homology are also referred to as the gene to be introduced.

The expression cassette of the present invention is such that a nucleotides sequence which forms a complementary base pair with a gene [e.g. trnG(tRNA-Gly(GCC)), trnV(tRNA-Val(GAC)), trnfM(tRNA-fMet(CAU)), rbcL gene, accD gene, trnI(tRNA-Ile (GAU)) and trnA(tRNA-Ala(UGU)), 3′rps12 (ribosomal protein S12 exon-3) gene, trnV(tRNA-Val(GAC)) etc.] sequence of a chloroplast DNA is added to 5′- and 3′-side of the gene to be introduced, so that the cassette is assuredly introduced into chloroplast DNA by homologous recombination. A nucleotide sequence forming a complementary base pair can be preferably used as long as it is a sequence having a nucleotide sequence of about 500 to 1500, which has a homologous part forming a complementary base pair with a gene of chloroplast DNA. Examples of such nucleotide sequence include a sequence which is substantially the same as that of a gene of chloroplast DNA, a sequence which is substantially the same as a partial sequence of a gene of chloroplast DNA, or a nucleotide sequence complementary to a sequence containing a sequence which is substantially the same as that of a gene of chloroplast DNA.

In addition, the nucleotide sequence is not limited to the aforementioned gene sequence of chloroplast DNA as long as it has a nucleotide sequence of about 1000 to 1500 from a position in which a gene to be introduced has been introduced, and forms a complementary base pair with a gene (e.g. trnG, trnfM, rbcL gene, accD gene, trnI, trnA, 3′rps12 gene, trnV etc.) of chloroplast DNA.

In this regard, it is necessary that a nucleotide sequence of chloroplast DNA is not changed except that a foreign gene is introduced. A nucleotide sequence of chloroplast DNA into which a foreign gene is introduced has been already registered in NCBI database, and is disclosed (registration number: NC 001879). A position at which a gene to be introduced is introduced in a chloroplast DNA is preferably between trnG and trnfM, between rbcL gene and accD gene, between trnI and trnA, and between 3′rps12 gene and trnV of chloroplast DNA, and is preferably a non-coding region sufficiently a part from each gene. The sufficiently apart is at least 50 bases or more, preferably about 100 to 1000 bases, more preferably about 200 to 500 bases from agene. The non-coding region may be any non-coding region on a chloroplast DNA.

An expression cassette using the rbcL gene and the accD gene will be explained in detail below.

The rbcL gene constituting an expression cassette which enhances photosynthesis activity is the gene of Rubisco encoded in the chloroplast genome. Rubisco catalyzes a CO₂ fixing reaction (carboxylase reaction) which is an initial stage of CO₂ fixation reaction cycle (Calvin cycle) of photosynthesis, and is a key enzyme which is rate-limiting in metabolism in the cycle. The enzyme also catalyzes a reaction (oxygenase reaction) for fixing oxygen (O₂). As a rbcL gene derived from chloroplasts in the present invention, the rbcL gene derived from tobacco chloroplasts can be used preferably.

The accD gene constituting an expression cassette which enhances photosynthesis activity is a gene of acetyl CoA carboxylase encoded in the chloroplast genome. Acetyl CoA carboxylase is an enzyme involved in fatty acid synthesis in plants. As an accD gene derived from chloroplasts in the present invention, the accD gene derived from tobacco chloroplasts can be used preferably.

By using an expression cassette having a chloroplast-derived rbcL gene and a chloroplast-derived accD gene, a gene encoding a protein having FBPase and/or SBPase activities is easily integrated into a chloroplast by homologous recombination, and there is an advantage that an amount of expression of a protein having FBPase and/or SBPase activities is increased in chloroplasts.

In addition, it is not necessary to use the full length rbcL gene and accD gene. For example, those genes may be used as long as they have a sequence having a length of a base pair of about 1000 to 1500 on the rbcL gene side or accD gene side from a position into which a gene to be introduced is introduced, in a non-coding region between the rbcL gene and the accD gene, and being capable of homologous recombination with the rbcL gene or the accD gene.

In addition, it is preferable that an expression cassette for enhancing photosynthesis activity has a ribosome-binding site upstream of the translation initiation point of a DNA fragment which contains a gene encoding a protein having FBPase and/or SBPase activities. By placing the ribosome-binding site upstream of the DNA fragment, the protein can be highly expressed. The ribosome-binding site may be situated adjacent to and upstream of a translation initiation point of the gene encoding the protein, and it is preferably located about 7 to 11 bases upstream of the translation initiation point, further preferably about 9 bases upstream of the translation initiation point. Such ribosome-binding site is any nucleotide sequence as long as it has the known per se nucleotide sequence to which ribosomes can bind, and the SD sequence is preferable. The SD sequence is an abbreviation of the Shine-Dalgarno sequence, which is a segment consisting of 4 to 7 nucleotides, and its nucleotide sequence is a part or all of 5′-AGGAGGU-3′ (SEQ ID NO: 18).

It is preferable that the expression cassette for enhancing photosynthesis activity further has a plant cell-derived promoter upstream of the ribosome-binding site. The promoter may be adjacent to a ribosome-binding site, or may be situated about 1 to 30 bases upstream, as long as it is located upstream of the ribosome-binding site. Examples of the promoter include a promoter of an elongation factor 1α gene (EF1α promoter), a 35S promoter, a psbA promoter, a PPDK promoter, a PsPAL1 promoter, a PAL promoter, a UBIZM1 ubiquitin promoter and a rrn promoter. Inter alia, a chloroplast-derived promoter is preferable, and a tobacco chloroplast-derived promoter is more preferable, and particularly, the tobacco chloroplast-derived psbA promoter described, for example, in SEQ ID NO: 7 of Sequence Listing can be used preferably.

It is preferable that an expression cassette for enhancing photosynthesis activity has a plant-derived terminator between a DNA fragment comprising a gene encoding a protein having FBPase and/or SBPase activities and the accD gene. The terminator may be adjacent to the DNA fragment, or may be situated about 1 to 30 bases downstream, as long as it is situated downstream of the DNA fragment. Examples of the terminator include a 35S terminator, a rps16 terminator, a CaMV35S terminator, an ORF25polyA transcription terminat a PsbA terminator. Inter alia, a chloroplast-derived terminator is preferable, a tobacco chloroplast-derived terminator is more preferable, the tobacco chloroplast-derived rps16 terminator is most preferable, and the tobacco chloroplast-derived rps16 terminator described, for example, in SEQ ID NO: 8of Sequence Listing can be used preferably.

In addition, it is preferable that an expression cassette has a gene for screening transformants. The gene for screening transformants is not particularly limited, and the known per se gene may be used. Examples of such gene include various drug resistance genes (aadA), and a gene compensating for auxotrophy of a host. More specific examples include an ampicillin resistance gene, a neomycin resistance gene (G418 resistant), a chloramphenicol resistance gene, a kanamycin resistance gene, a spectinomycin resistance gene, a URA3 gene and the like. More specifically, for example, a spectinomycin resistance gene described in SEQ ID NO: 9of Sequence Listing can be used preferably. In addition, it is preferable that a promoter for recognizing the gene (hereinafter, abbreviated as aadA promoter) and a terminator of the gene (hereinafter, abbreviated as aadA terminator) are disposed upstream and downstream of the gene, respectively. As the aadA promoter and aadA terminator, the aforementioned plant-derived promoter and terminator can be preferably used, and the rrn promoter and the psbA terminator are particularly preferable. An aadA promoter/aadA/aadA terminator is referred to as aadA cassette in some cases.

It is preferable that an aadA cassette for screening transformants is disposed between a rbcL gene and a promoter upstream of the ribosome-binding site.

It is preferable that an expression cassette used in the vector of the present invention is constructed in an order of the rbcL gene, the aadA cassette, the promoter, the ribosome-binding site, the DNA fragment comprising a gene encoding a protein having FBPase and/or SBPase activities, the terminat the accD gene from the 5′ side. Respective DNAs may be consecutive, or an intron sequence, for example, may be inserted between respective DNAs.

A recombinant gene vector of the present invention can be prepared, for example, by the following steps.

A first step is a step of making a pLD6 vector. Such vector can be easily made by the method described in Example [step 1]. A total nucleotide sequence of pLD6 is shown in SEQ ID NO: 10. In pLD6, a construction gene group is inserted into the cleavage site created by the NotI and SalI digestion of pLD6. The construction gene group has (a) a group consisting of a multicloning region (located from 3698 to 3748 in SEQ ID NO: 10) having a nucleotide sequence represented by SEQ ID NO: 11, the tobacco chloroplast-derived psbA promoter (located from 3569 to 3701 in SEQ ID NO: 10) represented by SEQ ID NO: 7 upstream therefrom, and the tobacco chloroplast-derived rps16 terminator (located from 3755 to 3913 in SEQ ID NO: 10) represented by SEQ ID NO: 8 downstream of the multicloning region, and, upstream of the group, (b) aadA gene (located from 2369 to 3173 in SEQ ID NO: 10) which is the spectinomycin resistance gene represented by SEQ ID NO: 9 as the gene for screen transformants, the tobacco chloroplast-derived rrn promoter (located from 2227 to 2368 in SEQ ID NO: 10) represented by SEQ ID NO: 12 upstream of the aadA gene, and a tobacco chloroplast-derived psbA terminator (located from 3175 to 3564 in SEQ ID NO: 10) represented by SEQ ID NO: 13 downstream of the aadA gene. The gene encoding a protein having FBPase and/or SBPase activities is inserted between restriction enzyme recognition sites (BglII, SphI, ClaI and EcoRI) of the aforementioned multicloning region. More specifically, for example, the gene encoding a spinach-derived SBPase represented by SEQ ID NO: 2 or the gene encoding spinach-derived FBPase represented by SEQ ID NO: 4, or the gene encoding a cyanobacterium-derived FBP/SBPase represented by SEQ ID NO: 6 is inserted into the cleavage site created by the SphI and EcoRI digestion of the multicloning region of the pLD6 vector. In this case, the nucleotide sequence at 13 to 17 positions of SEQ ID NO: (5′-aggag-3′) corresponds to the SD sequence, and functions as the ribosome-binding site. Hereinafter, a construction gene group in which the gene encoding a protein having FBPase and/or SBPase activities is inserted is referred to as FBP/SBP gene group, and the pLD6 vector in which the gene group is inserted is referred to as pLD6-FBP/SBP.

Then, pLD6-FBP/SBP is introduced into an appropriate host cell, and such host cell is cultured for cloning a FBP/SBP gene group.

A host cell can be appropriately selected from the known per se host cells, and examples thereof include prokaryotic organism such as Escherichia and Bacillus, eukaryotic organism such as yeast and filamentous fungus, plant cell or animal cell and the like. Condition for culturing a host cell may be according to the condition which is normally performed in the art, depending on a kind of the host cell. In addition, whether the gene encoding a protein having FBPase and/or SBPase activities has been successfully introduced into a cloned gene or not can be easily determined based on a selective marker, etc. possessed by pLD6-FBP/SBP and the like.

The next step is making the pLD200 vector. Such vector can be easily prepared by the method described in Example [step 2]. A FBP/SBP gene group is excised, using NotI and SalI, from a recombinant gene which has been cloned using pLD6-FBP/SBP in the previous step, and the excised gene group is inserted between cleavage sites of NotI and SalI of the polylinker of pLD200. A total nucleotide sequence of pLD200 is described in SEQ ID NO: 14. The polylinker has a nucleotide sequence (located from 2125 to 2145 of SEQ ID NO: 14) represented by SEQ ID NO: 17, and has a plurality of restriction enzyme sites (NotI, NheI and SalI). The pLD200 vector is a vector characterized in that it has an expression cassette comprising the polylinker, the tobacco chloroplast-derived rbcL gene (located from 423 to 1856 in SEQ ID NO: 14) having a nucleotide sequence represented by SEQ ID NO: 15 upstream therefrom, and the tobacco chloroplast-derived accD gene (located from 2624 to 3328 in SEQ ID NO: 14) having a nucleotide sequence represented by SEQ ID NO: 16 downstream therefrom. Thus, the pLD200 vector in which a FBP/SBP gene group is inserted is referred to as pLD200-FBP/SBP.

The aforementioned vector may be also obtained by inserting a polylinker (preferably, a gene having a nucleotide sequence represented by SEQ ID NO: 17) having a plurality of restriction enzyme sites, the tobacco chloroplast-derived rbcL gene upstream of the polylinker, and the tobacco chloroplast-derived accD gene downstream of the polylinker into to the known per se cloning vectors.

The thus prepared pLD200-FBP/SBP is introduced into a host cell to prepare a transformant. Thereupon, as a host cell, a plant cell is preferable, chloroplasts are more preferable, and tobacco chloroplasts are further more preferable. In this way, by using a plant cell, particularly chloroplasts, as a host cell, there is an advantage that the protein encoded by the introduced gene can be highly expressed, and flying of the introduced gene into environment via pollen can be prevented.

As a method of introducing pLD200-FBP/SBP into a host cell, particularly, chloroplasts to perform transformation, the known methods may be used. Examples of such methods include a particle gun method in which the expression vector is dusted with extremely fine particles of gold or tungsten, and the particles to which the expression vector are adhered are shot into a host cell with a gunpowder or a high pressure gas to introduce the expression vector. Inter alia, it is preferable to use a procedure by a particle gun (Svab, Z., Hajdukiewicz, P., and Maliga, P., Proc. Natl. Acad. Sci. USA, 1990, vol. 87, p. 8526-8530), or a procedure by PEG (Golds, T., Maliga, P., and Koop, H.-U., Bio/Technol., 1993, vol. 11, p. 95-97), in a system of introducing a gene into higher plant chloroplasts.

A plant having the aforementioned transformed chloroplasts of the present invention can be obtained by the known per se methods. Herein, the plant is not particularly limited, but higher plants are preferable, and a plant of which chloroplast transformation system is established is more preferable, including, for example, tobacco, rice, potato, rape and lettuce, and a tobacco is especially preferable. Examples of tobacco include Nicotiana acuminate, Nicotiana alata, Nicotiana attenuata, Nicotiana clevelandii, Nicotiana excelsior, Nicotiana forgetiana, Nicotiana gossei, Nicotiana glauca, Nicotiana glutinosa, Nicotiana langsdorffii, Nicotiana longiflora, Nicotiana obtusifolia, Nicotiana paniculata, Nicotiana plumbagifolia, Nicotiana quadrivalvis, Nicotiana repanda, Nicotiana rustica, Nicotiana sanderae, Nicotiana suaveolens, Nicotiana sylvestris, Nicotiana tabacum, Nicotiana tomentosa, Nicotiana tomentosiformis and the like. Inter alia, Nicotiana rustica and Nicotiana tabacum are preferable. In particular, Nicotiana tabacum is preferable and, among Nicotiana tabacum, “Burley”, “Yellow (Virginia)”, “Native” and “Oriental” are particularly preferable.

The aforementioned plants can be grown under the known per se condition depending on the plant.

Procedures of the genetic engineering or biotechnology can be easily performed by the methods described in commercially available experimental documents, for example, Molecular Cloning, Cold Spring Harbor Laboratory published in 1982, or Molecular Cloning, 2nd ed., Cold Spring Harbor Laboratory published in 1989, etc.

Vectors pLD6 and pLD200 utilized in a process of constructing a vector for introducing a gene into the tobacco chloroplast genome of the present invention are published in Japanese Patent Application No. 2001-083569.

The present invention will be explained in more detail by way of specific Example described below, but the present invention is not particularly limited thereto.

The meanings of respective abbreviations used in Example are as follows:

S.7942: Synechococcus PCC 7942

LB medium: Luria-Bertani medium

NaCl: sodium chloride

### EXAMPLE

### Preparation of Recombinant Gene

[Step 1] Preparation of pLD6-S.7942FBP/SBPase

A S.7942FBP/SBPase gene (fbp/sbp) represented by SEQ ID NO: 2 of Sequence Listing was inserted between restriction enzymes SphI and EcoRI sites of a vector pLD6 having the psbA promoter (PpsbA) by which high expression can be expected in tobacco chloroplasts, to prepare pLD6-S.7942FBP/SBPase. This pLD6-S.7942FBP/SBPase was introduced into Escherichia coli according to a conventional method. This Escherichia coli was cultured at 37° C. for 16 hours in LB medium supplemented with spectinomycin to select the Escherichia coli in which such gene was introduced. The selected Escherichia coli was cultured under the similar condition, cells were collected by centrifugation, and pLD6-S.7942FBP/SBPase (plasmid DNA) was purified by a conventional method. The LB medium includes 10 g of tryptone, 5 g of yeast extract, and 5 g of NaCl per liter. [Step 2] Preparation of pLD200-S.7942FBP/SBPase

The pLD6-S.7942FBP/SBPase purified in the step 1 was treated with restriction enzymes NotI and SalI, and then the fragment containing S.7942FBP/SBPase was inserted between NotI and SalI sites of the vector pLD200 for transforming chloroplasts which contains a part of the rbcL gene and a part of the accD gene of the tobacco chloroplast genome upstream of NotI and downstream of SalI, to prepare pLD200-S.7942FBP/SBPase. This pLD200-S.7942FBP/SBPase was introduced into Escherichia coli according to a conventional method. This Escherichia coli was cultured at 37° C. for 16 hours in LB medium supplemented with spectinomycin to select the Escherichia coli in which such gene was introduced. The selected Escherichia coli was cultured under the similar condition, cells were collected by centrifugation, and pLD200-S.7942FBP/SBPase (plasmid DNA) was purified according to a conventional method (FIG. 1).

[Step 3] Preparation of Chloroplast Transformant

The purified pLD200-S.7942FBP/SBPase was introduced into tobacco chloroplasts with a particle gun to prepare a chloroplast transformant. The transformation of tobacco chloroplasts was carried out according to the known method (Svab, Z., Hajdukiewicz, P. and maliga, P., Stable transformation of plastids in higher plants. Proc. Natl. Acad. Sci. USA, 87, 8526-8530 (1990)).

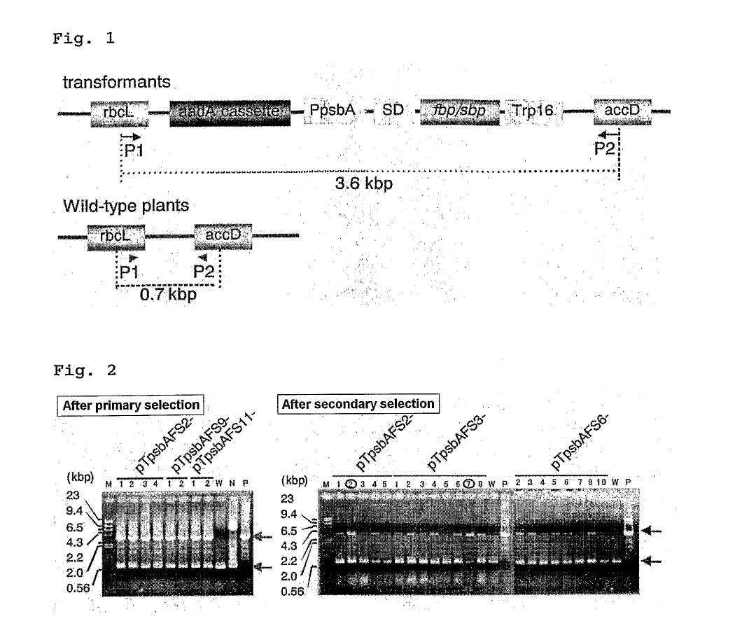

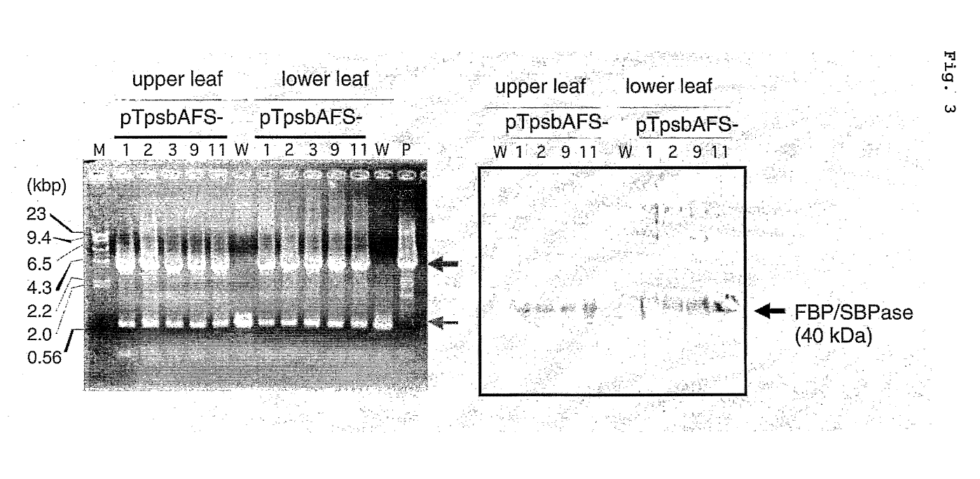

After redifferentiation on a spectinomycin-supplemented medium, a transformant (pTpsbAFS) 6 strain wherein S.7942FBP/SBPase was introduced into the chloroplast genome could be obtained by PCR. Also in T₁ generation produced by self hybridization, defect of the gene was not recognized (FIG. 2). Western blotting was performed using an anti-S.7942FBP/SBPase antibody and, as a result, the signal was recognized at a position of about 40 kDa coinciding with a molecular mass of S.7942FBP/SBPase only in the transformed plant (pTpsbAFS), and it was made clear that FBP/SBPase was highly expressed (FIG. 3).

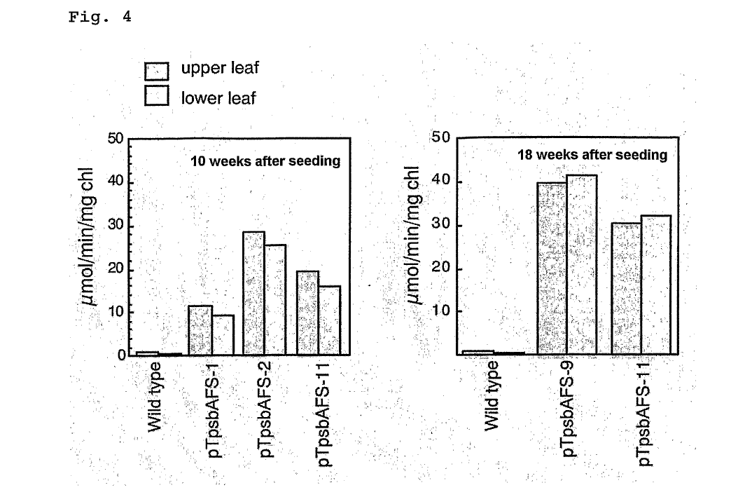

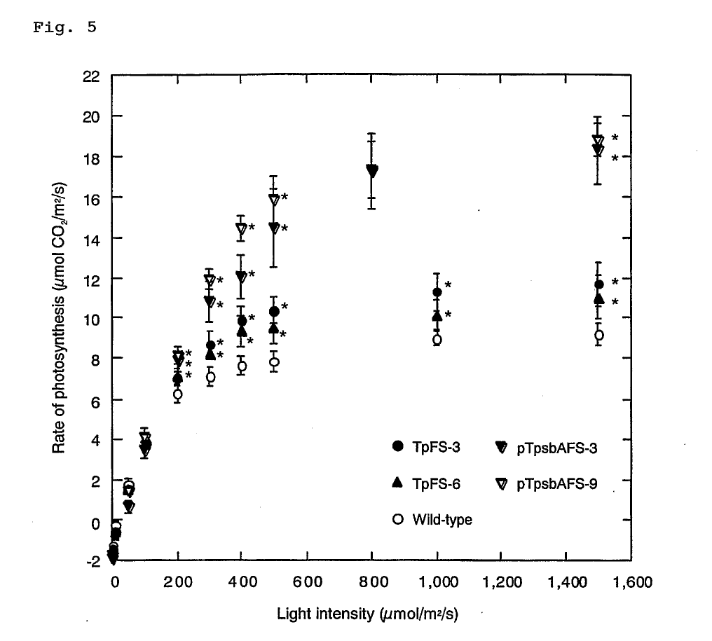

Using plants of 10 weeks and 18 weeks after seeding, FBPase activity was measured. The transformed plant had about 10 to 40-fold higher FBPase activity as compared with the wild strain (FIG. 4).

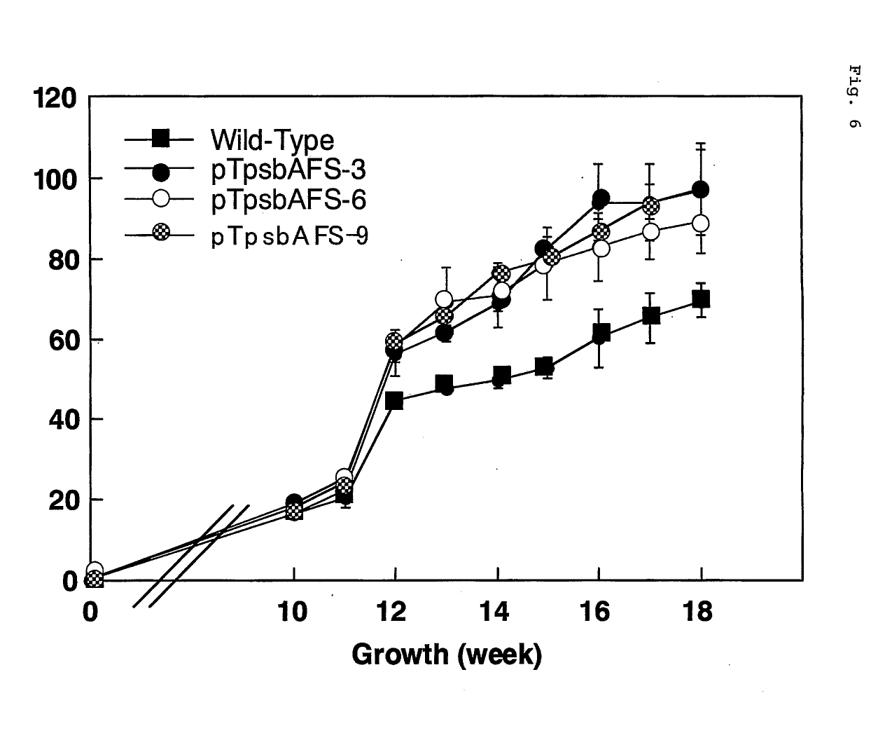

Using a T₁ generation 12 weeks after seeding, photosynthesis activity was measured by a change in light intensity under condition of the CO₂ concentration of 360 ppm. Results are shown in FIG. 5. Transformants (pTpsbAFS-3 and pTpsbAFS-9) and the wild strain (Wild-type) had a maximum photosynthesis rate at light intensity of about 500 μmol/m²/s and, thereafter, the rate was maintained. The photosynthesis rate of the transformant at a maximum was about 2-fold that of the wild strain.

For comparison, according to the method described in JP-A No. 2000-253768, the plasmid linked to S.7942FBP/SBPase was introduced into Agrobacterium tumefaciens LBA4404 to make a transformant (TpFS-3 and TPFS-6) infected with a leaf disk of tobacco. The TpFS-3 and TPFS-6 had an about 1.2 to 1.3-fold photosynthesis rate at a maximum as compared with a wild strain, which was far lower than the photosynthesis rates of pTpsbAFS-3 and pTpsbAFS-9. This demonstrates that the transformant of the present invention has an enhanced photosynthesis activity as compared with the wild strain and the transformed plant obtained by the conventional methods.

Furthermore, pTpsbAFS-3 and pTpsbAFS-9 showed a photosynthesis rate equivalent to a maximum of the wild strain at light intensity of about 200 μmol/m²/s, and a photosynthesis rate equivalent to a maximum of TpFS-3 and TPFS-6 at 300 μmol/m²/s. This demonstrates that the transformed plant of the present invention has sufficient photosynthesis activity even when light intensity is low.

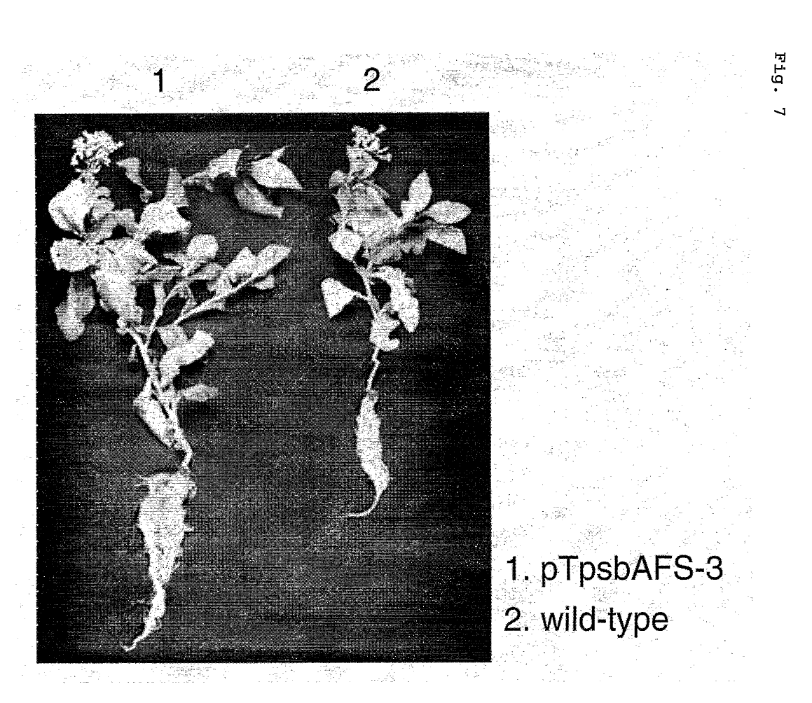

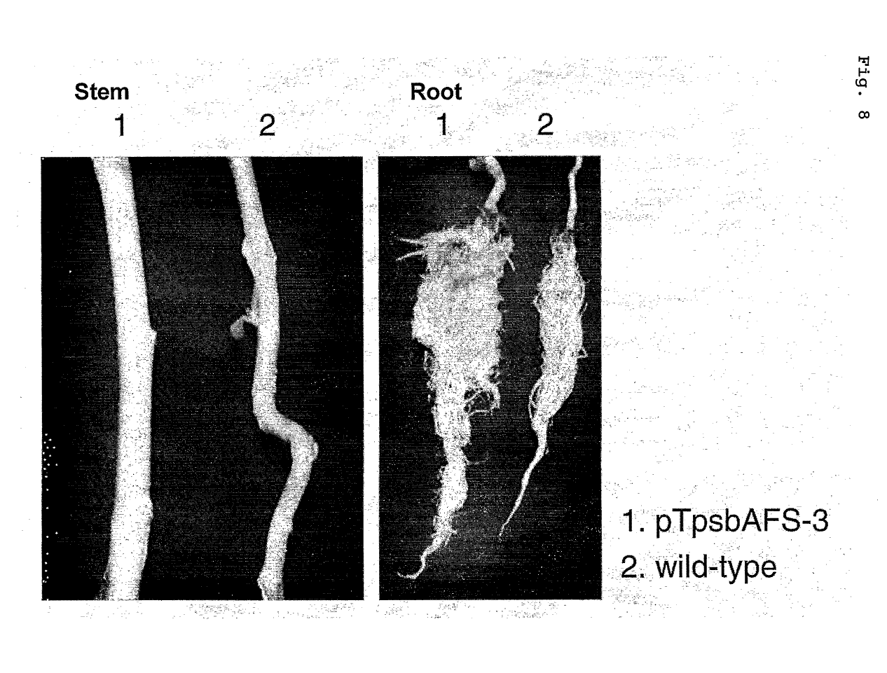

When growth of the transformants and growth of the wild strain were compared 18 weeks after seeding, growth of the transformed plants was clearly promoted as compared with the wild strain, and the final growth reached 1.2 to 1.3-fold that of a wild strain (FIGS. 6 and 7). In addition, a stem of a transformant was thicker than that of a wild strain, and also a root was remarkably developed (FIG. 8). Further, after 18 weeks, transformants had grown to be about 1.5-fold the size of a wild strain.

As mentioned above, by introducing a S.7942FBP/SBPase gene into the tobacco chloroplast genome, photosynthesis ability of tobacco leaves could be enhanced. Further, thereby, it becomes possible to promote growth, and increase the yield.

With respect to plants other than tobacco, a plant cell into which the S.7942FBP/SBP gene can be introduced and expressed can be prepared by introducing the aforementioned plasmid pLD200-S.7942FBP/SBP into chloroplasts with a particle gun, and selecting a resistant cell in a medium supplemented with spectinomycin.

For example, a transformed plant cell can be prepared by discharging the aforementioned plasmid with a particle gun into rape seed leaf, potato leaf blade, lettuce leaf blade, rice leaf blade or embryonic stem cell, and selecting a resistant cell on a spectinomycin-supplemented medium with an appropriate concentration. The resultant cells in which the S.7942FBP/SBP gene is introduced and expressed are redifferentiated under appropriate conditions, thereby to produce a plant having improved photosynthesis ability which promotes the growth. The transformation conditions for rape are described in Transgenic Research, 12(1), p. 111-114 (2003), those for potato in Plant Journal, 19(2), p. 209-216 (1999), those for rice in Nature Biotechnology, 17(9) p. 910-915 (1999) and those for lettuce in Sympodium of Japanese Society for Plant Cell and Molecular Biology, 1Da-10, 2004.

Similarly, with respect to other plant species, a transformed plant can be produced by discharging the aforementioned pLD200-S.7942FBP/SBP gene into a leaf blade or an embryonic stem cell with a particle gun, selecting a resistant cell on the spectinomycin-supplemented medium and redifferentiating the selected cell. The selection conditions using spectinomycin can be easily determined by observation of the growth and redifferentiation in the medium supplemented with various concentrations of spectinomycin. Usually, it is preferred to select a condition wherein a wild type strain cannot grow at a concentration as low as possible. The condition for redifferentiation of a callus into a plant can be determined by the conventional technique. For example, selection is carried out using a matrix medium containing auxin or cytokinin with a stepwise varied concentration, and optimum conditions for redifferentiation are determined. If required, gibberellin or amino acids may be added in some cases. Redifferentiation conditions from a callus in a variety of plant species into plants have been determined today. When the aforementioned chloroplast transformation technique is applied to these plants, there can be obtained a plant wherein the S.7942FBP/SBP gene has been introduced and expressed and which has an improved photosynthesis ability promoting the growth. With respect to plants redifferentiation conditions of which have not been established so far, when such redifferentiation becomes possible in the future, application of the vector of the present invention to such plants for introduction and expression of the S.7942FBP/SBP gene should make it possible to produce a plant whose growth is promoted due to its improved photosynthesis ability.

### INDUSTRIAL APPLICABILITY

A plant transformed using the gene recombinant of the present invention has high photosynthesis activity, and is useful as a quickly growing plant or a high yield plant.

1. A gene recombination vector containing an expression cassette for enhancing photosynthesis activity, comprising a DNA fragment comprising a gene encoding a protein having FBPase and/or SBPase activities between a Rubisco large subunit gene and an acetyl CoA carboxylase subunit gene.

2. The vector as claimed in claim 1, wherein the protein having FBPase activity is any one of the followings;
(a) a protein comprising an amino acid sequence described in SEQ ID NO: 1 of Sequence Listing;
(b) a protein comprising an amino acid sequence in which one or several amino acids are deleted, substituted, added or inserted in SEQ ID NO: 1 of Sequence Listing, and having FBPase activity; and
(c) a protein having at least 60% or more homology to an amino acid sequence described in SEQ ID NO: 1 of Sequence Listing, and having FBPase activity.

3. The vector as claimed in claim 1, wherein the gene encoding a protein having FBPase activity is a gene comprising any one of the following DNAs;
(a) DNA comprising a nucleotide sequence described in SEQ ID NO: 2 of Sequence Listing;
(b) DNA comprising a nucleotide sequence in which one or several bases are deleted, substituted, added or inserted in SEQ ID NO: 2 of Sequence Listing, and encoding a protein having FBPase activity;
(c) DNA which hybridizes with DNA comprising a nucleotide sequence complementary to DNA comprising a nucleotide sequence described in SEQ ID NO: 2 of Sequence Listing under stringent conditions, and comprises a nucleotide sequence encoding a protein having FBPase activity; and
(d) DNA having at least 60% or more homology to DNA comprising a nucleotide sequence described in SEQ ID NO: 2 of Sequence Listing, and comprising a nucleotide sequence encoding a protein having FBPase activity.

4. The vector as claimed in claim 1, wherein the protein having SBPase activity is any one of the following proteins;
(a) a protein comprising an amino acid sequence described in SEQ ID NO: 3 of Sequence Listing;
(b) a protein comprising an amino acid sequence in which one or several amino acids are deleted, substituted, added or inserted in SEQ ID NO: 3 of Sequence Listing, and having SBPase activity; and
(c) a protein having at least 60% or more homology to an amino acid sequence described in SEQ ID NO: 3 of Sequence Listing, and having SBPase activity.

5. The vector as claimed in claim 1, wherein the gene encoding a protein having SBPase activity is a gene comprising any one of the following DNAs;
(a) DNA comprising a nucleotide sequence described in SEQ ID NO: 4 of Sequence Listing;
(b) DNA comprising a nucleotide sequence in which one or several bases are deleted, substituted, added or inserted in SEQ ID NO: 4 of Sequence Listing, and encoding a protein having SBPase activity;
(c) DNA which hybridizes with DNA comprising a nucleotide sequence complementary to DNA comprising a nucleotide sequence described in SEQ ID NO: 4 of Sequence Listing under stringent conditions, and comprises a nucleotide sequence encoding a protein having SBPase activity; and
(d) DNA having at least 60% or more homology to DNA comprising a nucleotide sequence described in SEQ ID NO: 4 of Sequence Listing, and comprising a nucleotide sequence encoding a protein having SBPase activity.

6. The vector as claimed in claim 1, wherein the protein having FBPase and SBPase activities is any one of the followings:
(a) a protein comprising an amino acid sequence described in SEQ ID NO: 5 of Sequence Listing;
(b) a protein comprising an amino acid sequence in which one or several amino acids are deleted, substituted, added or inserted in SEQ ID NO: 5 of Sequence Listing; and having FBPase and SBPase activities; and
(c) a protein having at least 60% or more homology to an amino acid sequence described in SEQ ID NO: 5 of Sequence Listing; and having FBPase and SBPase activities.

7. The vector as claimed in claim 1, wherein the gene encoding a protein having FBPase and SBPase activities is a gene comprising any one of the following DNAs;
(a) DNA comprising a nucleotide sequence described in SEQ ID NO: 6 of Sequence Listing;
(b) DNA comprising a nucleotide sequence in which one or several bases are deleted, substituted, added or inserted in SEQ ID NO: 6 of Sequence Listing, and encoding a protein having FBPase and SBPase activities;
(c) DNA which hybridizes with DNA comprising nucleotide sequence complementary to a DNA comprising a nucleotide sequence described in SEQ ID NO: 6 of Sequence Listing under stringent conditions, and comprises a nucleotide sequence encoding a protein having FBPase and SBPase activities; and
(d) DNA having at least 60% or more homology to DNA comprising a nucleotide sequence described in SEQ ID NO: 6 of Sequence Listing, and comprising a nucleotide sequence encoding a protein having FBPase and SBPase activities.

8. The vector as claimed in claim 1, wherein the expression cassette has a ribosome-binding site upstream of a translation initiation point of a DNA fragment comprising a gene encoding a protein having FBPase and/or SBPase activities.

9. The vector as claimed in claim 8, wherein the expression cassette has a promoter upstream of a ribosome-binding site, and a terminator downstream of DNA fragment comprising a gene encoding a protein having FBPase and/or SBPase activities.

10. The vector as claimed in claim 9, wherein the promoter and the terminator are a promoter and a terminator derived from tobacco chloroplasts, respectively.

11. The vector as claimed in claim 1, wherein the Rubisco large subunit gene and the acetyl CoA carboxylase subunit gene are genes derived from tobacco, respectively.

12. A recombinant gene vector comprising an expression cassette containing a DNA fragment comprising a gene encoding a protein having FBPase and/or SBPase activities between a tobacco-derived Rubisco large subunit gene and an acetyl CoA carboxylase subunit gene, having a ribosome-binding site upstream of a translation initiation point of the DNA fragment, having a tobacco-derived promoter between a Rubisco large subunit gene and a ribosome-binding site, and having a tobacco-derived terminator between the acetyl CoA carboxylase subunit gene and the DNA fragment.

13. A transformed chloroplast characterized in that the vector according to claim 1 is introduced into chloroplasts.

14. A plant containing transformed chloroplasts according to claim 13.

15. The plant as claimed in claim 14, wherein the plant is tobacco.

16. A plant having 2-fold or higher FBPase activity compared to the original one, characterized in that a FBPase/SBPase gene is introduced into the chloroplast genome of higher plants and expressed using a chloroplast transformation technique.

17. A plant having two-fold or higher enhanced photosynthesis rate as compared with the wild variety, characterized in that a FBPase/SBPase gene is introduced into the chloroplast genome of higher plants using a vector according to claim 1, followed by expression.
